(19) United States
(12) Patent Application Publication
(10) Pub. No.: US 2025/0185967 A1
(43) Pub. Date: Jun. 12, 2025

(54) BIOLOGICAL INFORMATION MEASURING DEVICE

(71) Applicant: OMRON HEALTHCARE Co., Ltd., Kyoto (JP)

(72) Inventors: Kentaro MORI, Kyoto (JP); Yoshihide TOKKO, Kyoto (JP); Masaki HARADA, Kyoto (JP); Takaaki OKANISHI, Kyoto (JP); Kosuke ABE, Kyoto (JP); Shuhei MUNEHIRA, Kyoto (JP); Takeshi KUBO, Kyoto (JP)

(21) Appl. No.: 19/058,919

(22) Filed: Feb. 20, 2025

Related U.S. Application Data
(63) Continuation of application No. PCT/JP2023/037765, filed on Oct. 18, 2023.

(30) Foreign Application Priority Data
Feb. 2, 2023 (JP) 2023-014808

(51) Int. Cl. A61B 5/28; A61B 5/00; A61B 5/0205; A61B 5/021; A61B 5/022; A61B 5/251

(52) U.S. Cl. CPC A61B 5/28; A61B 5/0205; A61B 5/02141; A61B 5/02225; A61B 5/251; A61B 5/6824; A61B 2560/0468; A61B 2562/125; A61B 2562/227

(57) ABSTRACT

A biological information measuring device that is used by being attached to an arm portion of a human body and that is capable of measuring at least an electrocardiographic waveform includes an electrode member including a shaft portion on which screw processing is performed, and a head portion, wherein the head portion of the electrode member is fixed in a state of being in contact with an outer surface of a main body case, the shaft portion is joined to a sensor substrate in the main body case and electrically connected to the sensor substrate, and thus positioning of the sensor substrate in the main body case and fixation and electrical connection of the electrode and the sensor substrate can be performed together.

### CROSS-REFERENCE TO RELATED APPLICATIONS

This application is the U.S. national stage application filed pursuant to 35 U.S.C. 365 (c) and 120 as a continuation of International Patent Application No. PCT/JP2023/037765, filed Oct. 18, 2023, which application claims priority to Japanese Patent Application No. 2023-014808, filed Feb. 2, 2023, which applications are incorporated herein by reference in their entireties.

### TECHNICAL FIELD

The present invention belongs to the technical field related to healthcare, and particularly relates to a biological information measuring device.

### BACKGROUND ART

It has recently become common for an individual to measure information (hereinafter, also referred to as biological information) related to a body and health of the individual such as a blood pressure value and an electrocardiographic waveform on a daily basis by himself/herself by using a measuring device and utilize the measurement result for health management. For this reason, the demand for devices where importance is placed on portability has been increasing, and many portable measuring devices have been proposed (for example, Patent Document 1).

Patent Document 1 discloses a wristwatch-type biological information measuring device that includes an electrocardiographic electrode and that can measure an electrocardiographic waveform. Patent Document 1 discloses a structure in which an electrode disposed on the back surface side of a main body case (i.e., the side in contact with a skin) is provided with a protrusion toward the inside of the main body case, the protrusion is inserted into the main body case through an opening portion provided in the main body case, and the protrusion is electrically connected to a substrate at which an electric circuit is disposed via a conductive spring member (spring pin) in the main body case. With such a configuration, it is possible to measure an electrocardiographic waveform by disposing the electrode in contact with the human body.

### CITATION LIST

### Patent Literature

Patent Document 1: Chinese Patent No. 110794666

### SUMMARY OF INVENTION

### Technical Problem

Here, in the configuration described in Patent Document 1, the protrusion of the electrode and the substrate are electrically connected by the spring member. However, when the electrode is electrically connected by the spring member, the contact (electrical connection) between the electrode and the substrate may become unstable due to the stress of the spring member. In addition, since the electrode and the substrate are connected via the spring member, the relative position of the substrate and/or the electrode with respect to the main body case in the vertical direction becomes unstable. As a result, the electrode in contact with the human body may wobble, and appropriate measurement may not be performed.

On the other hand, it is also conceivable to position the device main body and the substrate by using a mechanism for fixing the substrate to the case (screw fastening or double-sided tape). However, in the case of screw fastening, an extra area is also required at the mounting surface of the substrate, and in the case of double-sided tape, the double-sided tape may be peeled off due to the stress of the spring.

On the other hand, it is also conceivable that the electrode and the substrate are fixed while being electrically connected by soldering without using the spring member. However, even in this case, an area for soldering is required on the substrate. Further, in consideration of contact with the human body, the electrode is preferably made of a material having rust resistance and safety, such as stainless steel, but stainless steel cannot be soldered with typical solder, which increases cost.

When it is acceptable to increase the area of the substrate, adopting a mechanism such as screwing for the spring contact causes no big problem. However, in the case of a wearable terminal, it is necessary to achieve both reduction in size and weight in order to improve attachability as much as possible and measurement of a plurality of pieces of biological information (multifunctionality). For this reason, it is desirable to reduce the size and space of each component, in addition to the area of the substrate, as much as possible.

In view of the above-described problems, an object of the present invention is to provide a technology that can improve the reliability of fixing an electrode and a substrate to a case of a biological information measuring device having an electrocardiogram measuring function, and that can contribute to space saving of the device.

### Solution to Problem

A biological information measuring device according to the present invention adopts the following configurations in order to solve the above problems. That is, the biological information measuring device according to the present invention is a biological information measuring device that is used by being attached to an arm portion of a human body and that is capable of measuring at least an electrocardiographic waveform, the biological information measuring device including
a main body case including
a bottom portion located on a side in contact with the arm portion in an attachment state, and
a front surface located on a side opposite to the bottom portion,
an electrode member including a contact surface exposed at the bottom portion such that the contact surface is contactable with the arm portion, and a first sensor substrate disposed inside the main body case in a vicinity of the bottom portion, the first sensor substrate including an opening portion provided with an electrode contact at an outer periphery, the electrode contact electrically connecting the first sensor substrate and the electrode member, wherein
the electrode member includes
a shaft portion constituting at least a part of a joining mechanism joining the electrode member and the first sensor substrate, the shaft portion extending in a first direction toward the arm portion in an attachment state in which the biological information measuring device is attached to the arm portion, and
a head portion having an area larger than an area of the shaft portion when viewed from the first direction, the contact surface being formed at the head portion,
the electrode member is joined to the first sensor substrate by the joining mechanism via the opening portion and is electrically connected to the first sensor substrate via the electrode contact or a part of the joining mechanism in contact with the electrode contact, and
a surface of the head portion opposite to the contact surface is fixed in a state of being in contact with an outer surface of the bottom portion of the main body case, and thus arrangement of the first sensor substrate in the main body case is determined.

Note that, in the above description, the head portion and the main body case may be fixed by bonding with an adhesive or by insert molding. Examples of the joining mechanism can include a screw, a rivet, and a pin. With the configuration described above, the electrode member and the sensor substrate are positioned with respect to the main body case and fixed, and the electrode member and the sensor substrate can be electrically connected. For this reason, it is not necessary to separately provide a configuration for electrical connection, and it is possible to improve the reliability of fixing the electrode and the substrate to the case of the device and to contribute to space saving of the device.

In addition, screw processing may be performed on the shaft portion, and the electrode member may be screwed with a screw member constituting a part of the joining mechanism via the opening portion and thus joined to the first sensor substrate, and the shaft portion or the screw member may be brought into contact with the electrode contact, and thus the electrode member may be electrically connected to the first sensor substrate. That is, the joining mechanism may be the shaft portion (screw processing performed thereon) and the screw member. Further, the screw member may be a male screw, and the shaft portion may be formed in a cylindrical shape, an inner wall of the shaft portion may be threaded, and thus the shaft portion may function as a female screw.

In addition, a recessed portion may be formed at the bottom portion of the main body case in a shape in which the head portion is fittable, and the head portion may be fixed in a state of being fitted to the recessed portion. With such a configuration, the contact surface of the electrode member can be formed to be flush at the bottom portion of the case without the head portion protruding from the outer surface of the bottom portion of the case. Further, since the movement of the head portion in the direction orthogonal to the first direction is also restricted, it is possible to more stably position and fix the head portion and the first sensor substrate.

Further, the electrode member may be insert-molded in the main body case, the head portion of the electrode member may be provided with a retaining protrusion portion having an area larger than an area of the contact surface when viewed from an axial direction, and a retaining recessed portion engaged with the retaining protrusion portion may be formed at an inner wall of the recessed portion of the bottom portion. With such a configuration, since the movement of the head portion toward the side away from the main body case is also regulated, it is possible to more stably position and fix the head portion and the first sensor substrate.

Further, the contact surface of the electrode member may be formed in a circular shape when viewed from an axial direction. With such a configuration, it is possible to suppress a variation in rotation at the time of insertion or bonding at the time of manufacturing the device. As a result, a high yield can be achieved at the time of manufacturing.

In addition, at least a part of the bottom portion may be provided with a light-transmissive portion, a sensor substrate housing portion may be provided inside the main body case in a region including the light-transmissive portion when viewed from the first direction, and a sensor substrate including a light emitting element and a light receiving element may be housed in the sensor substrate housing portion. Here, the “sensor substrate” may be the first sensor substrate described above or may be a substrate different from the first sensor substrate. With such a configuration, it is possible to provide a multifunctional biological information measuring device capable of measuring biological information (for example, pulse wave, pulse, blood oxygen saturation, and blood pressure) using a photoelectric sensor.

When the sensor substrate is a substrate different from the first substrate, a sensor substrate set may be housed in the sensor substrate housing portion, the sensor substrate set including the first sensor substrate and a second sensor substrate, the second sensor substrate being located closer to the bottom portion than the first sensor substrate being to the bottom portion, the second sensor substrate being electrically connected to the first sensor substrate, the second sensor substrate including a light emitting element and a light receiving element. The first sensor substrate and the second sensor substrate may be electrically connected by a spring contact. With such a configuration, the repulsive force of the spring contact acts between the first sensor substrate and the second sensor substrate that are positioned, which can stabilize electrical connection between both substrates and stably fix the second sensor substrate to the inner wall of the main body case on the bottom portion side.

The biological information measuring device may include a blood pressure measuring mechanism including a cuff configured to measure a blood pressure of the human body, wherein the sensor substrate housing portion may be provided at a center portion of the main body case when viewed from the first direction, and a joining portion joining the cuff and the main body case may be provided in at least a part of a region of the bottom portion where the sensor substrate housing portion is not provided when viewed from the first direction. In addition, a cuff cover configured to fix the cuff to the main body case may be provided in a region of the bottom portion, the region being a region where the sensor substrate housing portion is not provided and that includes the joining portion when viewed from the first direction.

When a blood pressure measuring mechanism using an oscillometric method is further added, it is necessary to provide a contact portion with the cuff on the bottom portion side of the main body case, and thus it is necessary to save the space of the sensor substrate housing portion at the bottom portion. For this reason, the technology of achieving fixation and electrical connection between the electrode member and the sensor substrate with one configuration as in the present invention is suitable for the configuration described above.

The blood pressure measuring mechanism may include a piezoelectric pump, a valve, and a flow path plate forming a flow path of gas, the flow path plate may be formed of a conductive material and disposed closer to the front surface than the first sensor substrate is to the front surface in the main body case, and the first sensor substrate and the flow path plate may be electrically connected by a spring contact. With such a configuration, the GND area of the first sensor substrate can be increased, and noise resistance can be improved. In addition, since the flow path plate is formed of a conductive material, the flow path plate can have a function of shielding the sensor substrate set against noise from the internal devices such as the piezoelectric pump.

In addition, the sensor substrate housing portion may be formed to protrude toward the arm portion in the attachment state. Such a shape is preferable because the quality of biological information obtained by various sensors such as an electrocardiographic electrode a photoelectric sensor can be improved as the degree of contact with the human body increases.

The configurations and processing described above can be combined with one another to constitute the present invention unless the combination leads to technical contradiction.

### Advantageous Effects of Invention

According to the present invention, it is possible to provide a technology that can improve the reliability of fixing an electrode and a substrate to a case of a biological information measuring device having an electrocardiogram measuring function, and that can contribute to space saving of the device.

### BRIEF DESCRIPTION OF DRAWINGS

FIG. 1 is an external perspective view illustrating an outline of a biological information measuring device according to an embodiment of the present invention.

FIG. 2 is a side view illustrating the outline of the biological information measuring device according to the embodiment.

FIG. 3 is an explanatory view illustrating an arrangement relationship when the biological information measuring device according to the embodiment is attached to a wrist.

FIG. 4 is an external view when a main body portion of the biological information measuring device according to the embodiment is viewed from a bottom portion side.

FIG. 5 is a schematic cross-sectional view when the biological information measuring device according to the embodiment is viewed from a side.

FIG. 6 is a schematic cross-sectional view of the vicinity of a sensor substrate housing portion of the biological information measuring device according to the embodiment.

FIG. 7(A) is a schematic cross-sectional view for describing connection between electrodes and a sensor substrate of the biological information measuring device according to the embodiment. FIG. 7(B) is an explanatory view for describing an electrode member according to the embodiment. FIG. 7(C) is an explanatory view of configurations of opening portions of a first sensor substrate according to the embodiment.

FIG. 8 is a block diagram illustrating a functional configuration of the biological information measuring device according to the embodiment.

FIG. 9(A) is an explanatory view of a first modified example of the embodiment.

FIG. 9(B) is an explanatory view of a second modified example of the embodiment.

### DESCRIPTION OF EMBODIMENTS

### First Embodiment

An embodiment of the present invention will be specifically described below with reference to the drawings. However, it should be noted that the dimensions, material, shape, relative arrangement, and the like of components described in the embodiment below are not intended to limit the scope of this invention thereto alone, unless otherwise stated.

### Device Configuration

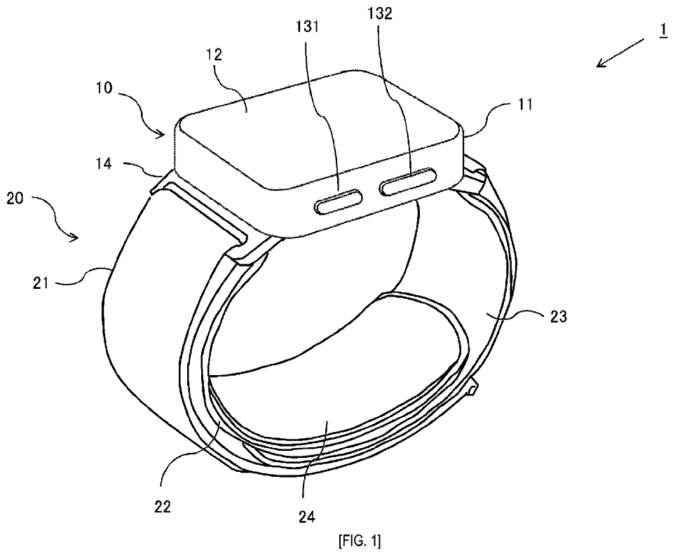

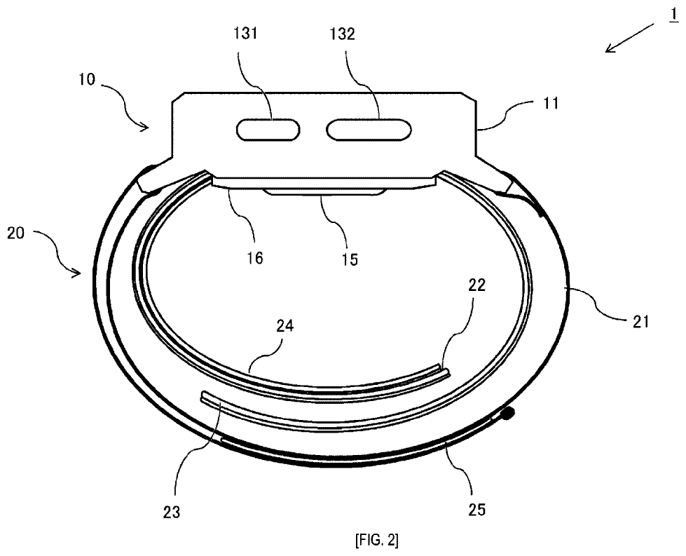

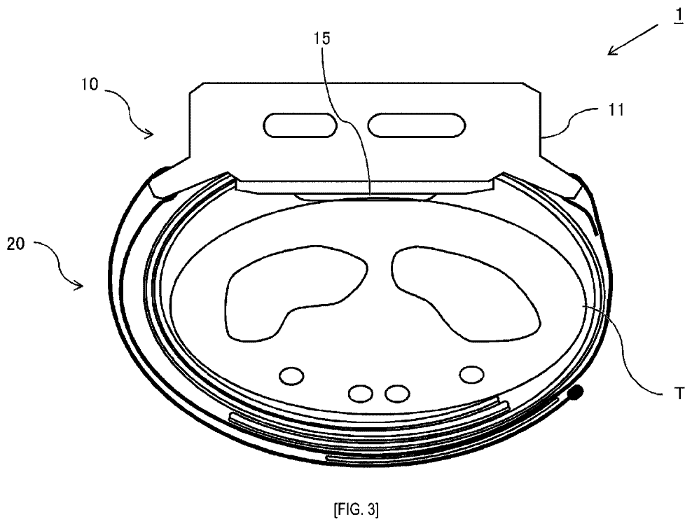

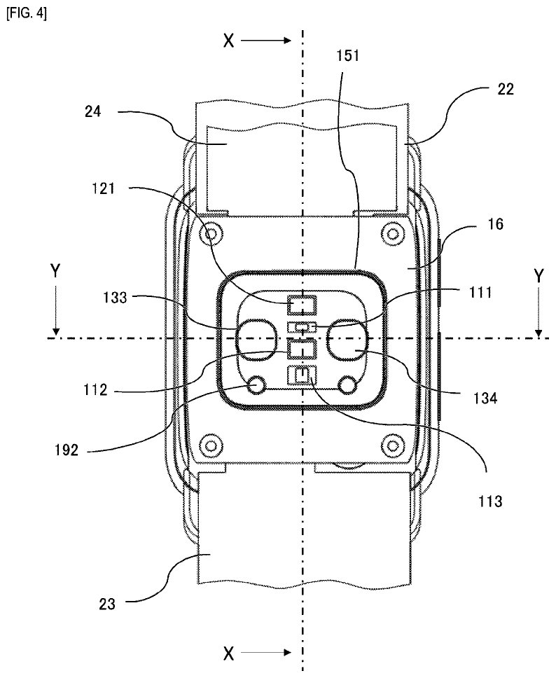

FIG. 1 is an external perspective view illustrating an outline of a configuration of a biological information measuring device 1 according to the present embodiment. FIG. 2 is a side view illustrating the outline of the configuration of the biological information measuring device 1 according to the present embodiment. As illustrated in FIGS. 1 and 2, the biological information measuring device 1 is generally a wristwatch-type wearable device including a main body portion 10 and a belt portion 20, and can measure biological information such as a pulse wave (pulse), a blood pressure value, and an electrocardiographic waveform in a state of being attached to a wrist T of a human body. FIG. 3 illustrates an arrangement relationship between the wrist T and components of the biological information measuring device 1 according to the present embodiment when the biological information measuring device 1 is attached to the wrist T.

As illustrated in FIGS. 1 and 2, the main body portion 10 includes a main body case 11 and a cuff cover 16 described below. The main body case 11 is provided with a display 12 (for example, an organic EL display or the like), operation buttons 131 and 132, a lug 14, and the like, and is also provided with a sensor substrate housing portion 15 in which a sensor substrate is housed. Note that, in the present embodiment, a side on which the display 12 is formed is referred to as a front surface of the main body case 11, and a side on which the sensor substrate housing portion 15 is formed is referred to as a bottom portion of the main body case 11. In the following description, a front surface side of the main body case 11 may be referred to as an upper side, and a bottom portion side of the main body case 11 may be referred to as a lower side. Note that, in the present embodiment, the operation buttons 131 and 132 are formed of a conductor and also function as electrodes for electrocardiographic waveform measurement.

FIG. 4 is an external view of the main body portion 10 when viewed from the bottom portion side. As illustrated in FIG. 4, the bottom portion of the main body case 11 includes a central region covered with a light-transmissive resin cover 151, and a region that corresponds to the outer periphery of the central region and that is covered with the cuff cover 16. The inner side of the main body case 11 in the region covered with the resin cover 151 corresponds to the sensor substrate housing portion 15. The sensor substrate housing portion 15 is located in the central region of the main body case 11 in plan view and is covered with the light-transmissive resin cover 151 at the bottom portion. Moreover, as illustrated in FIGS. 2 and 3, the sensor substrate housing portion 15 is formed so as to protrude toward the wrist T relative to the cuff cover 16 in the attachment state.

In addition, a first electrode 133 and a second electrode 134 are provided at the bottom portion of the main body case 11 such that contact surfaces with the human body are exposed. One of the first electrode 133 and the second electrode 134 functions as a GND electrode during electrocardiographic waveform measurement. During the electrocardiographic waveform measurement, the biological information measuring device 1 is attached, the contact surfaces of the first electrode 133 and the second electrode 134 are brought into contact with the skin surface at the attachment portion, and an operation button is touched with a finger on the side where the biological information measuring device 1 is not attached. Thus, the electrocardiographic waveform measurement can be performed by I-induction. Note that the detailed structures of the first electrode 133 and the second electrode 134 will be described below.

In addition, a charging terminal 192 is provided at the bottom portion of the main body case 11. A rechargeable battery (not illustrated in FIG. 4) can be charged by connecting a connection terminal of a power supply device and the charging terminal 192.

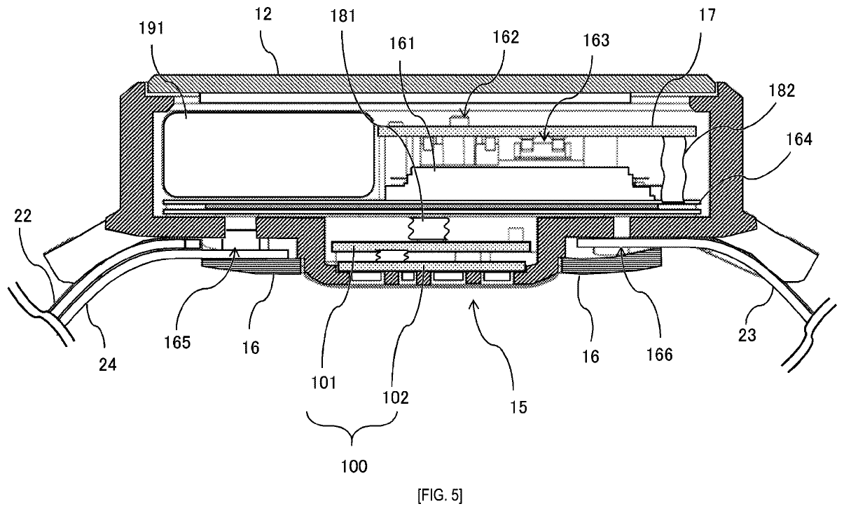

As illustrated in FIG. 5, a first LED 111, a second LED 113, a first photodiode (PD) 112, and a second PD 121 mounted at the lower surface (mounting surface) of the second sensor substrate 102 described below can be seen through the resin cover 151 from the bottom portion side of the main body case 11. These configurations will be described in detail below.

The belt portion 20 includes a belt 21 and a surface fastener 25 for fixing the biological information measuring device 1 to the wrist T, and also includes a first pressing cuff 22 and a second pressing cuff 23 for compressing an artery in the wrist T, and a sensing cuff 24 for detecting a pressure pulse wave. Note that connection portions between the cuffs 22, 23, and 24, and the main body case 11 are covered with the cuff cover 16. The cuff cover 16 protects the connection portions between the cuffs 22, 23, and 24, and the main body case 11, and also has a function of fixing the cuffs 22, 23, and 24 to the main body case 11.

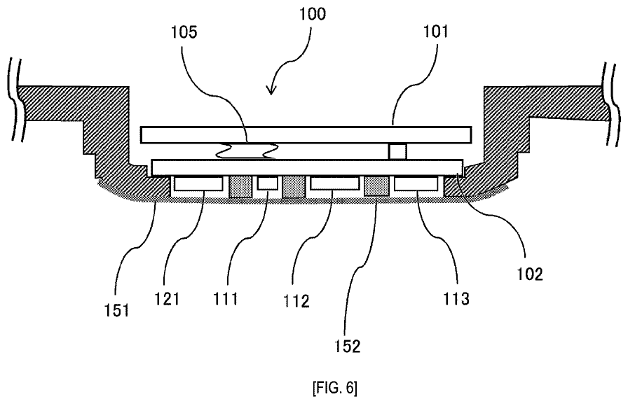

Next, the internal configuration of the main body case 11 will be described with reference to FIGS. 5 and 6. FIG. 5 is a schematic cross-sectional view corresponding to an X-X cross-section of FIG. 4. FIG. 6 is an enlarged view of the vicinity of the sensor substrate housing portion 15 in FIG. 5. Note that FIGS. 5 and 6 are not accurate cross-sectional views, and the configuration is appropriately omitted and deformed for convenience of description. As illustrated in FIG. 5, a rechargeable battery 191, a control board 17, a piezoelectric pump 161, a valve 162, a pressure sensor 163, a flow path plate 164, and the like are housed inside the main body case 11. In addition, the sensor substrate housing portion 15 formed in a protrusion shape is provided in the vicinity of the bottom portion of the main body case 11, and a sensor substrate set 100 including a first sensor substrate 101 and a second sensor substrate 102 is housed in the sensor substrate housing portion 15.

The bottom portion of the main body case 11 is provided with a first connection portion 165 and a second connection portion 166 in a part of a region where the sensor substrate housing portion 15 is not provided in plan view. The first connection portion 165 connects the main body case 11 (more specifically, the flow path plate 164 in the case) and the first pressing cuff 22 and the sensing cuff 24. The second connection portion 166 similarly connects the main body case 11 and the second pressing cuff 23. The first connection portion 165 and the second connection portion 166 are covered with the cuff cover 16 provided in the region corresponding to the outer periphery of the sensor substrate housing portion 15 at the bottom portion of the main body. In addition, as described above, the portion where the sensor substrate housing portion 15 is located is covered with the resin cover 151 made of a transparent resin.

As the rechargeable battery 191, a general-purpose rechargeable battery such as a lithium ion battery can be adopted, and the rechargeable battery 191 can be repeatedly charged by receiving power supply via the charging terminal 192. In addition, a processor such as a CPU, a memory such as a RAM, and the like (not illustrated) are mounted at the control board 17, and the control board 17 performs entire control of the biological information measuring device 1. The piezoelectric pump 161, the valve 162, the pressure sensor 163, the flow path plate 164, the first pressing cuff 22, the second pressing cuff 23, and the sensing cuff 24 are components related to blood pressure measurement. The flow path plate 164 is a conductive member (metal), and a flow path for feeding gas from the piezoelectric pump 161 to each cuff is formed therein.

The first sensor substrate 101 and the flow path plate 164 are electrically connected by a spring contact 181, and the control board 17 and the flow path plate 164 are also electrically connected by a spring contact 182. Since the first sensor substrate 101 and the control board 17 are electrically connected to the conductive flow path plate 164, it is possible to increase the GND areas of the first sensor substrate 101 and the control board 17 and to improve noise resistance. In addition, for the first sensor substrate 101, the flow path plate 164 also functions as a shield against noise generated from the internal devices such as the piezoelectric pump 161.

Next, the sensor substrate housing portion 15 and the sensor substrate set 100 will be described. As illustrated in FIG. 6, the sensor substrate housing portion 15 is a space that protrudes from the bottom portion of the main body case 11 toward the side in contact with the human body. The sensor substrate set 100 in which the first sensor substrate 101 and the second sensor substrate 102 are vertically stacked in two stages is housed in the space. Note that the first sensor substrate 101 and the second sensor substrate 102 are connected by a conductive spring contact 105, and these two substrates function as a pair.

At the lower surface of the second sensor substrate 102, two light emitting elements of the first LED 111 and the second LED 113, and two light receiving elements of the first photodiode (PD) 112 and the second PD 121 are provided. Note that, in the present embodiment, the first LED 111 emits green irradiation light, and the second LED 113 emits red light and/or infrared light in addition to green light. Isolation walls 152 are also provided so as to isolate the first LED 111, the second LED 113, the first PD 112, and the second PD 121 from each other.

On the other hand, although not illustrated, a capacitor, an amplifier circuit, an analog-to-digital (A/D) conversion circuit, and the like are mounted at the first sensor substrate 101. Note that the first sensor substrate 101 may be a double-sided mounting substrate. In this way, the sensor substrate set 100 has a vertically stacked two-stage structure including the second sensor substrate 102 and the first sensor substrate 101. Thus, compared to a case where all components are mounted at one substrate, it is possible to greatly reduce the area of the substrates in plan view.

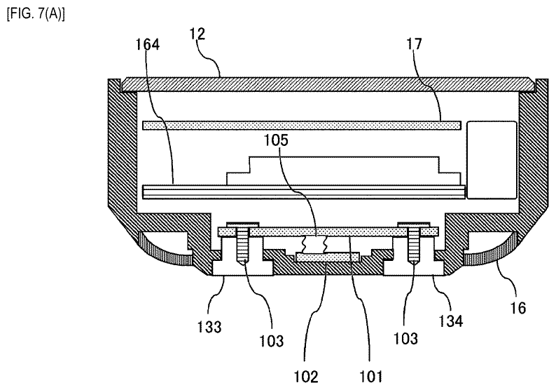

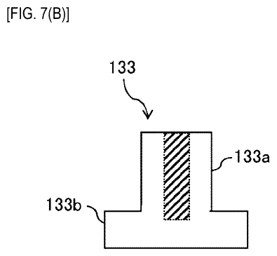

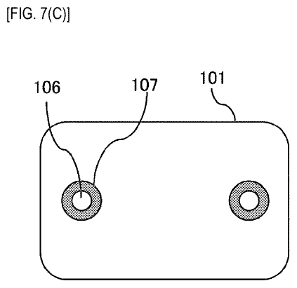

Next, a mode of connection between the first sensor substrate 101 and the electrodes will be described with reference to FIGS. 7(A), 7(B), and 7(C). FIG. 7(A) is a schematic cross-sectional view corresponding to a Y-Y cross-section of FIG. 4. However, FIG. 7(A) is not an accurate cross-sectional view, and the configuration is omitted and deformed for convenience of description. FIG. 7(B) is an explanatory view illustrating the structure of the first electrode 133. FIG. 7(C) is an explanatory view illustrating an outline of the lower surface of the first sensor substrate 101. As illustrated in FIG. 7(A), the first electrode 133 and the second electrode 134 are fixed in a state of being in contact with the lower surface of the first sensor substrate 101.

Here, referring to FIG. 7(B), the first electrode 133 has a so-called hat shape in a side view, and roughly includes a shaft portion 133a (corresponding to a crown portion of the hat) and a head portion 133b (corresponding to a brim portion of the hat). That is, the head portion 133b has an area larger than that of the shaft portion in plan view. Note that the surface of the head portion 133b on the side opposite to the shaft portion 133a serves as the contact surface in contact with the human body when the device is attached. The shaft portion 133a according to the present embodiment is formed in a hollow cylindrical shape, and the inner wall is threaded. That is, the shaft portion 133a functions as a female screw. Note that although the first electrode 133 has been described here, the same also applies to the second electrode 134.

As illustrated in FIG. 7(C), opening portions 106 are provided in the first sensor substrate 101, and an electrode pad 107 is formed on the outer periphery of each opening portion. As illustrated in FIG. 7(A), the first electrode 133 and the second electrode 134 are screwed with screw members 103 as male screws via the opening portions 106 of the first sensor substrate 101, so that the first electrode 133 and the second electrode 134 are fixed to the first sensor substrate 101. The fixing is performed in a state in which the distal end surfaces of the shaft portions of the first electrode 133 and the second electrode 134 are in contact with the electrode pads 107 formed at the outer peripheries of the opening portions 106 of the first sensor substrate 101. Thus, the first electrode 133 and the second electrode 134 are fixed to the first sensor substrate 101 in a state being electrically connected to the first sensor substrate 101.

Further, as illustrated in FIG. 7(A), the head portion of each of the first electrode 133 and the second electrode 134 is in a state of being fitted into a recessed portion formed in the main body case 11. More specifically, the surface of the head portion on the side where the shaft portion is provided is bonded and fixed in a state of being in contact with the outer surface of the recessed portion of the main body case 11. In this way, in the main body case 11, the arrangement of the first sensor substrate 101 joined by screw fastening to the first electrode 133 and the second electrode 134 fixed to the main body case 11 (relative positional relationship with respect to the main body case 11) is determined.

In addition, a force toward the bottom portion of the main body case 11 is applied to the first sensor substrate 101 by screw fastening. Here, since the first sensor substrate 101 is connected to the second sensor substrate 102 via the spring contact 105, the second sensor substrate 102 is pressed against the inner wall on the bottom portion side of the main body case 11. Accordingly, the second sensor substrate 102 is stably fixed to the main body case 11, and the first sensor substrate 101 and the second sensor substrate 102 are also stably connected.

### Functional Configuration of Device

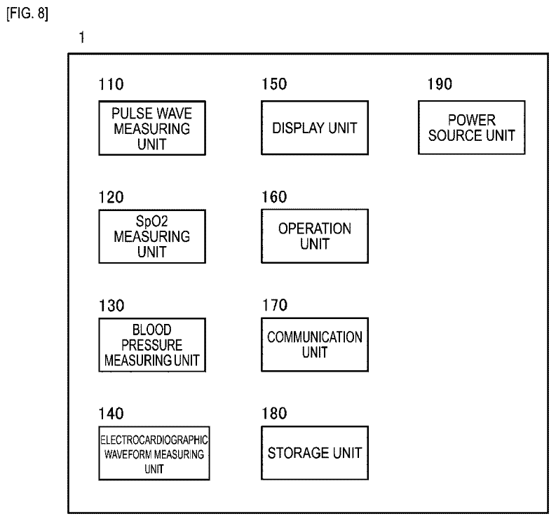

Next, a functional configuration of the biological information measuring device 1 will be described. FIG. 8 is a block diagram illustrating the functional configuration of the biological information measuring device 1. As illustrated in FIG. 8, the biological information measuring device 1 according to the present embodiment includes functional units of a pulse wave measuring unit 110, a blood oxygen saturation (SpO2) measuring unit 120, a blood pressure measuring unit 130, an electrocardiographic waveform measuring unit 140, a display unit 150, an operation unit 160, a communication unit 170, a storage unit 180, and a power source unit 190. The processor of the control board 17 reads a program from the memory and executes the program to control each configuration of the biological information measuring device 1, thereby implementing these functional units.

The pulse wave measuring unit 110 includes the first LED 111, the second LED 113, and the first PD 112, and measures a pulse wave by a so-called photoplethysmographic method to calculate a pulse. Specifically, green light is emitted from the first LED 111 and the second LED 113, and reflection light reflected in the living body is received by the first PD 112, so that a blood flow volume that changes with a heartbeat (change in volume of a blood vessel) is detected, and a pulse wave is measured.

The SpO2 measuring unit 120 includes the second LED 113 and the second PD 121, and the second PD 121 receives reflection light of red light or infrared light emitted from the second LED 113, so that the SpO2 measuring unit 120 measures blood oxygen saturation from the intensity of the reflection light.

The blood pressure measuring unit 130 includes the piezoelectric pump 161, the valve 162, the pressure sensor 163, the flow path plate 164, the first pressing cuff 22, the second pressing cuff 23, and the sensing cuff 24, and measures a blood pressure by a so-called oscillometric method. Since the blood pressure measurement by the oscillometric method is a well-known technology, a detailed description thereof will be omitted.

The electrocardiographic waveform measuring unit 140 includes the operation buttons 131 and 132, the first electrode 133 and the second electrode 134 provided at the bottom portion of the main body case 11, and an electrocardiographic waveform measuring circuit (not illustrated), and measures an electrocardiographic waveform by a so-called I-induction method. Specifically, an electrocardiographic waveform is measured based on a potential difference between the first electrode 133 and the second electrode 134 that are in contact with the wrist T of one arm in the attachment state, and a finger of the other hand in contact with the operation button 131 or 132 functioning as an electrode.

The display unit 150 includes the display 12, and displays various types of information such as a measurement result of biological information and a menu screen. The operation unit 160 includes the operation buttons 131 and 132, and receives an input operation by the user via these buttons. The communication unit 170 includes an antenna for wireless communication (not illustrated), and communicates with another electronic device such as an information processing terminal by, for example, BLE communication. Note that a terminal for wired communication may be provided.

The storage unit 180 includes a main storage device (not illustrated) such as a random access memory (RAM) and stores various types of information such as application programs and measured biological information. In addition to the RAM, for example, a long-term storage medium such as a flash memory may be provided. The power source unit 190 includes the rechargeable battery 191 and the charging terminal 192, and functions as a power supply source to each unit constituting the biological information measuring device 1.

### Effect of Present Embodiment

With the configuration of the biological information measuring device 1 according to the present embodiment, it is possible to stably fix and electrically connect the electrodes on the bottom portion side of the main body case 11 and the first sensor substrate 101. With such a configuration, contacts between the electrodes and the substrate of individual devices can be prevented from varying due to individual differences. That is, the yield at the time of manufacturing can be increased. In addition, the shaft portions of the first electrode 133 and the second electrode 134 have a predetermined length, and thus it is possible to position the first sensor substrate 101 at a position distant from the inner wall of the bottom portion of the main body case 11. With this configuration, it is possible to easily dispose another substrate closer to the bottom portion than the first sensor substrate 101 is. Therefore, substrates related to biological information acquisition are configured as the sensor substrate set 100 having a vertical two-stage configuration including the first sensor substrate 101 and the second sensor substrate 102, and it is easy to reduce the area of the substrates related to the sensor substrates in plan view.

### MODIFIED EXAMPLES

Note that, in the above-described embodiment, the recessed portions are formed at the bottom portion of the main body case 11, and the head portions of the first electrode 133 and the second electrode 134 are fitted to the recessed portions. However, the embodiment of the present invention is not limited to such an aspect. The head portions of the electrodes may have any shape as long as the electrodes and the first sensor substrate 101 can be positioned in the main body case 11. Hereinafter, modified examples of the shapes of the head portions of the electrodes and the bottom portion of the main body case 11 engaged with the head portions will be described. Note that, in the following description, components common to those of the biological information measuring device 1 according to the first embodiment are denoted by the same reference numerals, and detailed descriptions thereof will be omitted.

### Modified Example 1

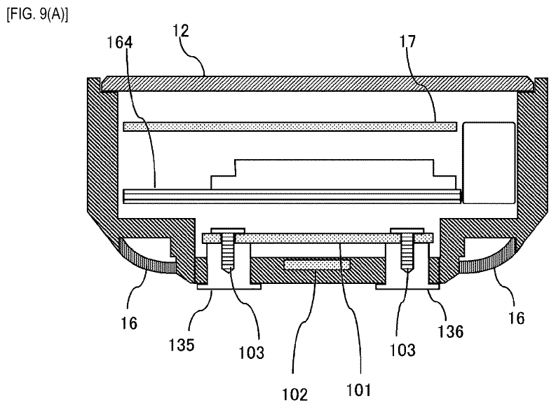

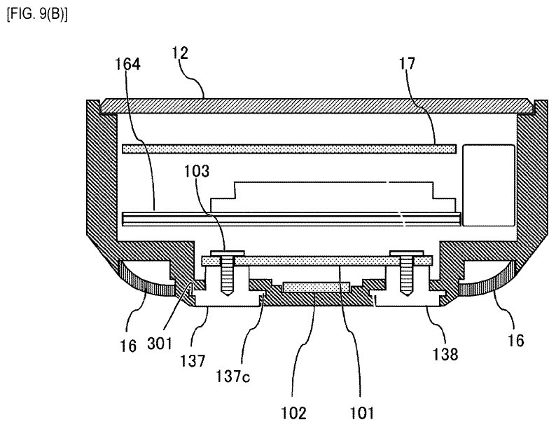

FIG. 9(A) is an explanatory view illustrating a first modified example, and illustrates the shapes of a first electrode 135, a second electrode 136, and the vicinity of a bottom portion of a main body case 11 according to the present modified example. As illustrated in FIG. 9(A), no recessed portion is formed in the bottom portion of the main body case 11 according to the present modified example, and a head portion of each of the first electrode 135 and the second electrode 136 is fixed to the main body case 11 in a state in which a contact surface is located at a position slightly protruding from the outer surface of the bottom portion of the main body case 11. Specifically, the surface of the head portion opposite to the contact surface may be bonded to the outer surface of the main body case 11 with an adhesive.

### Modified Example 2

FIG. 9(B) is an explanatory view illustrating a second modified example, and illustrates the shapes of a first electrode 137, a second electrode 138, and the vicinity of a bottom portion of a main body case 11 according to the present modified example. As illustrated in FIG. 9(B), a flange-shaped retaining protrusion portion 137c is further formed at a head portion of the first electrode 137 according to the present modified example. The retaining protrusion portion 137c is fixed in a state of being engaged with a retaining recessed portion 301 provided at a recessed inner wall of the bottom portion of the main body case 11. Note that although only the first electrode 137 has been described here, the same also applies to the second electrode. In the present modified example, the first electrode 137 and the second electrode 138 are insert-molded in the main body case 11, so that these electrodes are fixed. With such a configuration of the present modified example, since the movement of the head portion toward the side where the head portion comes off from the main body case 11 is regulated, it is possible to more stably position and fix the electrodes and the first sensor substrate 101.

### Other Points

The description of each example described above is merely illustrative of the present invention, and the present invention is not limited to the specific embodiment described above. Within the scope of the technical idea of the present invention, various modifications and combinations may be made. For example, the biological information measuring device is only required to include an electrode and a circuit for measuring an electrocardiographic waveform, and a function and a configuration for acquiring other biological information are not necessarily indispensable. Specifically, a configuration in which the second sensor substrate 102 is not provided, that is, a configuration in which not the sensor substrate set 100 but only a substrate corresponding to the first sensor substrate 101 is housed in the sensor substrate housing portion 15 may be employed.

Although the electrode pads 107 are provided at the lower surface of the first sensor substrate 101 in the above-described examples, the electrode pads may be provided at the upper surface of the first sensor substrate 101. In this case, for example, by using conductive materials for the screw members, it is possible to make electrical connection between the first sensor substrate 101 and the electrodes.

In the examples described above, the joining mechanism between each electrode and the first sensor substrate 101 is a male screw and a female screw, and the shaft portion of each electrode is a female screw. However, the shaft portion may be configured as a male screw, and the screw member may be configured as a female screw. That is, the electrode may be a bolt, and the screw member may be a nut.

Furthermore, the joining mechanism between each electrode and the first sensor substrate 101 is not necessarily limited to a screw. For example, by using a pin instead of the screw member and press-fitting the pin into a shaft portion formed in a cylindrical shape, it is possible to simultaneously perform bonding and electrical connection between the electrode and the first sensor substrate. Alternatively, an electrode pad may be provided on the upper side of the first sensor substrate, and the distal end of the shaft portion may be riveted so as to be in contact with the electrode pad, that is, the electrode member may be used like a rivet, so that the electrode and the first sensor substrate are joined and electrically connected.

In addition, in the above-described examples, the sensor substrate housing portion 15 is configured to protrude toward the human body at the bottom portion of the main body case 11, but this configuration is not necessarily required, and the bottom portion of the main body case 11 including the region of the sensor substrate housing portion 15 may have a flush structure. In addition, the position where the sensor substrate housing portion 15 is disposed in plan view is not necessarily in the vicinity of the center portion of the bottom portion.

### REFERENCE NUMERALS LIST

1 Biological information measuring device
10 Main body portion
11 Main body case
12 Display
14 Lug
15 Sensor substrate housing portion
16 Cuff cover
17 Control board
20 Belt portion
21 Belt
22 First pressing cuff
23 Second pressing cuff
24 Sensing cuff
25 Surface fastener
100 Sensor substrate set
101 First sensor substrate
102 Second sensor substrate
103 Screw member
105, 181, 182 Spring contact
106 Opening portion
107 Electrode pad
111 First LED
112 First PD
113 Second LED
121 Second PD
131, 132 Operation button
133, 135, 137 First electrode
134, 136, 138 Second electrode
137c Retaining protrusion portion
161 Piezoelectric pump
162 Valve
163 Pressure sensor
164 Flow path plate
165 First connection portion
166 Second connection portion
151 Resin cover
152 Isolation wall
191 Rechargeable battery
192 Charging terminal
301 Retaining recessed portion
T Wrist

1. A biological information measuring device that is used by being attached to an arm portion of a human body and that is capable of measuring at least an electrocardiographic waveform, the biological information measuring device comprising:
a main body case including
a bottom portion located on a side in contact with the arm portion in an attachment state, and
a front surface located on a side opposite to the bottom portion;
an electrode member including a contact surface exposed at the bottom portion such that the contact surface is contactable with the arm portion; and
a first sensor substrate disposed inside the main body case in a vicinity of the bottom portion, the first sensor substrate including an opening portion provided with an electrode contact at an outer periphery, the electrode contact electrically connecting the first sensor substrate and the electrode member, wherein
the electrode member includes
a shaft portion constituting at least a part of a joining mechanism joining the electrode member and the first sensor substrate, the shaft portion extending in a first direction toward the arm portion in an attachment state in which the biological information measuring device is attached to the arm portion, and
a head portion having an area larger than an area of the shaft portion when viewed from the first direction, the contact surface being formed at the head portion,
the electrode member is joined to the first sensor substrate by the joining mechanism via the opening portion and is electrically connected to the first sensor substrate via the electrode contact or a part of the joining mechanism in contact with the electrode contact, and
a surface of the head portion opposite to the contact surface is fixed in a state of being in contact with an outer surface of the bottom portion of the main body case, and thus arrangement of the first sensor substrate in the main body case is determined.

2. The biological information measuring device according to claim 1, wherein
screw processing is performed on the shaft portion, and the electrode member is screwed with a screw member constituting a part of the joining mechanism via the opening portion and thus joined to the first sensor substrate, and
the shaft portion or the screw member is brought into contact with the electrode contact, and thus the electrode member is electrically connected to the first sensor substrate.

3. The biological information measuring device according to claim 2, wherein
the screw member is a male screw, and
the shaft portion is formed in a cylindrical shape, an inner wall of the shaft portion is threaded, and thus the shaft portion functions as a female screw.

4. The biological information measuring device according to claim 1, wherein
a recessed portion is formed at the bottom portion of the main body case in a shape in which the head portion is fittable, and the head portion is fixed in a state of being fitted to the recessed portion.

5. The biological information measuring device according to claim 4, wherein
the electrode member and the main body case are formed by insert molding,
the head portion of the electrode member is provided with a retaining protrusion portion having an area larger than an area of the contact surface when viewed from an axial direction, and
a retaining recessed portion engaged with the retaining protrusion portion is formed at an inner wall of the recessed portion of the bottom portion.

6. The biological information measuring device according to claim 1, wherein
the contact surface of the electrode member is formed in a circular shape when viewed from an axial direction.

7. The biological information measuring device according to claim 1, wherein
at least a part of the bottom portion is provided with a light-transmissive portion,
a sensor substrate housing portion is provided inside the main body case in a region including the light-transmissive portion when viewed from the first direction, and
a sensor substrate including a light emitting element and a light receiving element is housed in the sensor substrate housing portion.

8. The biological information measuring device according to claim 1, wherein
at least a part of the bottom portion is provided with a light-transmissive portion,
a sensor substrate housing portion is provided inside the main body case in a region including the light-transmissive portion when viewed from the first direction,
a sensor substrate set is housed in the sensor substrate housing portion, the sensor substrate set including the first sensor substrate and a second sensor substrate, the second sensor substrate being located closer to the bottom portion than the first sensor substrate being to the bottom portion, the second sensor substrate being electrically connected to the first sensor substrate, the second sensor substrate including a light emitting element and a light receiving element, and
the first sensor substrate and the second sensor substrate are electrically connected by a spring contact.

9. The biological information measuring device according to claim 7, further comprising:
a blood pressure measuring mechanism including a cuff configured to measure a blood pressure of the human body, wherein
a joining portion joining the cuff and the main body case is provided in at least a part of a region of the bottom portion where the sensor substrate housing portion is not provided when viewed from the first direction.

10. The biological information measuring device according to claim 9, wherein
a cuff cover configured to fix the cuff to the main body case is provided in a region of the bottom portion, the region being a region where the sensor substrate housing portion is not provided and that includes the joining portion when viewed from the first direction.

11. The biological information measuring device according to claim 9, wherein
the blood pressure measuring mechanism includes a piezoelectric pump, a valve, and a flow path plate forming a flow path of gas,
the flow path plate is formed of a conductive material and is disposed closer to the front surface than the first sensor substrate is to the front surface in the main body case, and
the first sensor substrate and the flow path plate are electrically connected by a spring contact.

12. The biological information measuring device according to claim 7, wherein
the sensor substrate housing portion is formed to protrude toward the arm portion in the attachment state.
